(19) United States
(12) Patent Application Publication
(10) Pub. No.: US 2016/0199036 A1
(43) Pub. Date: Jul. 14, 2016

(54) C-Mode Ultrasound Image Data Visualization

(71) Applicant: ULTRASONIX MEDICAL CORPORATION, Richmond (CA)

(72) Inventors: Laurent Pelissier, Vancouver (CA); Reza Zahiri, Vancouver (CA); Bo Zhuang, Westminster (CA)

(73) Assignee: ULTRASONIX MEDICAL CORPORATION, Richmond (CA)

(21) Appl. No.: 14/912,626

(22) PCT Filed: Aug. 19, 2013
(86) PCT No.: PCT/IB2013/001797

§ 371 Date: Feb. 18, 2016

(51) Int. Cl. A61B 8/08; A61B 8/00

(52) U.S. Cl. CPC A61B 8/5207; A61B 8/466; A61B 8/483; A61B 8/4483; A61B 8/488

(57) ABSTRACT

An ultrasound imaging apparatus (100) includes a transducer array (102) configured to acquire a 3D plane of US data parallel to the transducer array. The transducer array includes a 2D array of transducer elements (104). The ultrasound imaging apparatus further includes a 3D US data processor (116) that visually enhances the structure of tissue of interest and extracts voxels representing tissue of interest therefrom. The ultrasound imaging apparatus further includes a display (118), located opposite the transducer array, that displays the extracted voxels representing the tissue of interest the 3D plane of US 3D US data.

### TECHNICAL FIELD

The following generally relates to ultrasound imaging and more particularly to C-mode ultrasound image data visualization.

### BACKGROUND

Ultrasound imaging provides useful information about interior characteristics of an object or subject. An ultrasound imaging apparatus has included at least a transducer array that transmits an ultrasound signal into an examination field of view. As the signal traverses structure therein, portions of the signal are attenuated, scattered, and/or reflected off the structure, with some of the reflections traversing back towards the transducer array. The later reflections are referred to as echoes. The transducer array receives the echoes.

In B-mode ultrasound imaging, the received echoes correspond to a two dimensional (2D) slice, which is perpendicular to the face of the transducer array, through the object or subject. The received echoes are processed to generate a two dimensional image of the slice, which can be displayed via a monitor display. A three-dimensional (3D) image can be created from a series of stacked adjacent 2D images. B-mode images have been combined with color flow, Doppler flow, and/or other information.

In Doppler-mode ultrasound imaging, the ultrasound signal is used to acoustically image flow. Generally, Doppler ultrasound employs the Doppler Effect to determine the direction of flow of a flowing structure and/or a relative velocity of the flowing structure such as blood cells flowing in vessels. The Doppler information can be visualized in a graph of velocity as a function of time, visualized as a color overlay superimposed over a B-mode and/or other image.

In C-mode ultrasound imaging, the received echoes correspond to a 2D volume, at a predetermined depth and thickness, which is parallel to the face of the transducer array and transverse to a B-mode image. Unfortunately, imaging vessels in C-mode may not be straight forward in that the user has to know where a vessel of interest is likely to be and how to orient the transducer array to scan the vessel. For example, angling the transducer array incorrectly may result in the loss of contact between the transducer array and the skin, which would result in loss of the image.

### SUMMARY

Aspects of the application address the above matters, and others.

The following relates to processing 3D ultrasound data acquired from a 2D array and displaying tissue of interest-only anatomy of the 3D ultrasound data in a 2D or 3D display. In one non-limiting instance, the 2D array is part of a device that includes an integrated display, integrated in a side of the device opposite the location of the transducer array, and the display effectively becomes a window for looking into the subject at the interest-only anatomy. With such a display, no specific training or hand-eye spatial coordination is required by the user to identify tissue of interest.

In one aspect, an ultrasound imaging apparatus includes a transducer array configured to acquire a 3D plane of US data parallel to the transducer array. The transducer array includes a 2D array of transducer elements. The ultrasound imaging apparatus further includes a 3D US data processor that visually enhances the structure of tissue of interest and extracts voxels representing tissue of interest therefrom. The ultrasound imaging apparatus further includes a display, located opposite the transducer array, that displays the extracted voxels representing the tissue of interest the 3D plane of US 3D US data.

In another aspect, a method includes obtaining C-mode 3D image data. The C-mode 3D image data includes voxels representing tissue of interest and other tissue (other than the tissue of interest). The method further includes filtering the C-mode 3D image data to visually enhance the tissue of interest. The method further includes segmenting the voxels representing the tissue of interest from the C-mode 3D image data. The method further includes projecting the segmented voxels onto a 2D surface or a 3D volume. The method further includes visually displaying the projected segmented voxels so that the tissue of interest appears adjacent to the display.

In another aspect, a computer readable storage medium is encoded with computer readable instructions. The computer readable instructions, when executed by a processor, causes the processor to: acquire 3D US imaging data with voxels representing tissue of interest and other tissue, wherein the 3D US imaging data is C-mode data, visually enhance the structure of tissue of interest through filtering, extract the voxels representing the tissue of interest from the 3D US imaging data, at least one of surface or volume render the extracted voxels, and register the rendered voxels with a 2D array the acquired the 3D US imaging data; and display the registered voxels.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings, in which like references indicate similar elements and in which:

FIG. 1 schematically illustrates an example ultrasound imaging system that includes a 3D US data processor;

FIG. 2 schematically illustrates an example of the 3D US data processor, with a tissue analyzing filter that can reconstruct and enhance the tissue of interest;

FIG. 3 schematically illustrates an example of the tissue of interest enhancer with B-mode and non-B-mode data enhancing;

FIG. 4 schematically illustrates an example of the tissue of interest enhancer with B-mode, non-B-mode, and Doppler data enhancing;

FIG. 5 schematically illustrates an example of the tissue of interest enhancer with B-mode and Doppler data enhancing;

FIG. 6 schematically illustrates an example of the tissue of interest enhancer with Doppler data enhancing; and

FIG. 7 illustrates an example ultrasound imaging method for visualizing 3D US data.

### DETAILED DESCRIPTION

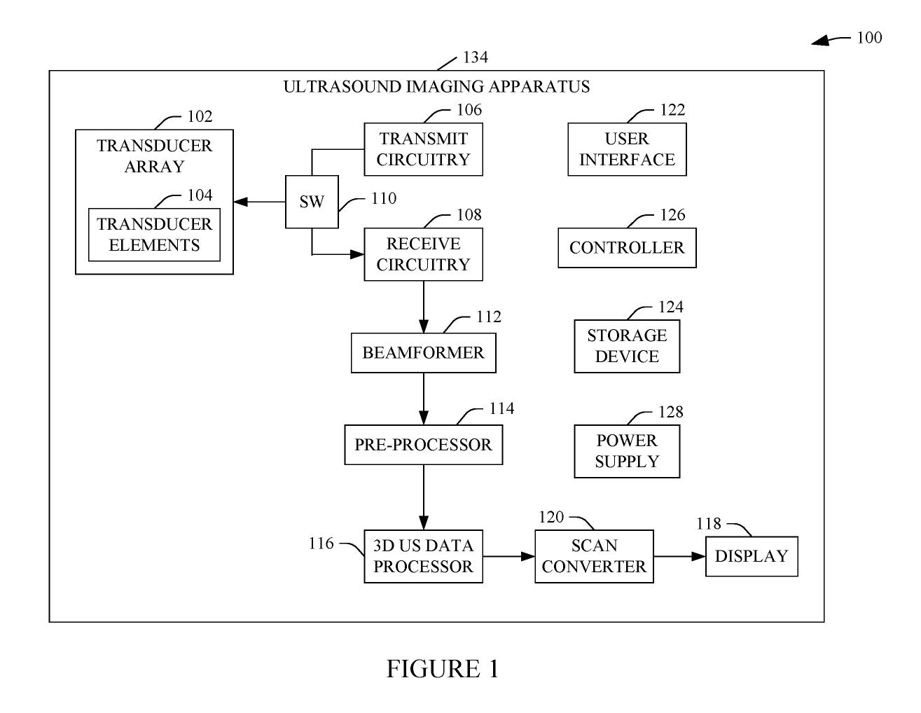

FIG. 1 schematically illustrates an imaging apparatus, such as an ultrasound (US) imaging apparatus 100.

A transducer array 102 includes a two-dimensional (2D) array of transducer elements 104. The transducer elements 104 convert electrical signals to an ultrasound pressured field and vice versa respectively to transmit ultrasound signals into a field of view and receive echo signals, generated in response to interaction with structure in the field of view, from the field of view. The transducer array 102 can be square, rectangular and otherwise shape, linear and/or curved, fully populated or sparse, etc. For example, the transducer array 102 may include a 32×32 array, a 64×64 array, a 16×32 array, and/or other array of the transducer elements 104.

Transmit circuitry 106 generates a set of pulses (or a pulsed signal) that are conveyed, via hardwire and/or wirelessly, to the transducer array 102. The set of pulses excites a set of the transducer elements 104 to transmit ultrasound signals. This includes signals in connection with 3D imaging such as C-Mode imaging. C-Mode imaging is discussed at least in U.S. Pat. No. 6,245,017 to Hashimoto et al., entitled “3D Ultrasonic Diagnostic Apparatus,” and filed Oct. 29, 1999, and other patents. The transducer 102 may be invoked to transmit signals for imaging a volume at a depth of approximately five (5.0) millimeter (mm) to approximately five (5.0) centimeter (cm) with respect to a surface of a subject in physical contact with the transducer array 102. The transmit circuitry 106 can also generate a set of pulses for B-mode, Doppler, and/or other imaging.

Receive circuitry 108 receives a set of echoes (or echo signals) generated in response to a transmitted ultrasound signal interacting with structure in the field of view. The receive circuitry 106 is configured to receive at least C-mode data and, optionally B-mode, Doppler, and/or other imaging data. A switch (SW) 110 controls whether the transmit circuitry 106 or transmit circuitry 108 is in electrical communication with the transducer elements 104. A beamformer 112 processes the received echoes by applying time delays to echoes, weighting echoes, summing delayed and weighted echoes, and/or otherwise beamforming received echoes, creating beamformed data. A pre-processor 114 processes the beamformed data. Suitable pre-processing includes, but is not limited to echo-cancellation, wall-filtering, basebanding, averaging and decimating, envelope detection, log-compression, FIR and/or IIR filtering, and/or other processing.

A 3D US data processor 116 processes the beamformed data, which includes beamformed 3D volumetric US imaging data. As described in greater detail below, the 3D US data processor 116 processes the beamformed data and can generate tissue of interest-only data (e.g., just a vessel of interest), which, when visually displayed in 2D or 3D via a display 118 of the apparatus 100 and/or other display, effectively renders the display 118 a window into a subject showing the tissue of interest-only data. For example, where the tissue of interest-only data is a vessel (e.g., a vein and/or an artery), the display 118 provides a window that visually shows the vessel, while non-vessel tissue is visually suppressed. It is to be appreciated that by doing so a user of the apparatus 100 does not require any specific training or hand-eye spatial coordination to orient the apparatus 100 to visualize vessels and/or other tissue of interest.

As will also be discussed herein, the 3D US data processor 116 may also generate B-mode images, Doppler images, and /or other images. The 3D US data processor 116 can be implemented via one or more processors (e.g., central processing unit (cpu), microprocessor, controller, etc.) executing one or more computer readable instructions encoded or embedded on computer readable storage medium, which excludes transitory medium, such as physical memory. Additionally or alternatively, an instruction can be carried by transitory medium, such as a carrier wave, a signal, and/or other transitory medium. The display 118 can be a light emitting diode (LED), liquid crystal display (LCD), and/or type of display.

A scan converter 120 converts the output of the 3D US data processor 116 to generate data for display, e.g., by converting the data to the coordinate system of the display 118. A user interface (UI) 122 includes an input device(s) (e.g., a physical button, a touch screen, etc.) and/or an output device(s) (e.g., a touch screen, a display, etc.), which allow for interaction between a user and the ultrasound imaging apparatus 100. A storage device 124 can be used to store data. A controller 126 controls one or more of the components 102-124. Such control can be based on a mode of operation (e.g., B mode, C-Mode, Doppler, etc.) and/or otherwise. A power source 128 includes a battery, a capacitor and/or other power storage device with power that can be supplied to the apparatus 100 to power one or more of the components therein, and/or receives power from an external power source such as an AC power supply (e.g., an AC electrical outlet or receptacle), a DC power supply, a battery charger, etc.

The US ultrasound imaging apparatus 100 can be part of a hand-held ultrasound imaging apparatus 134, as shown in FIG. 1. An example of such an apparatus is described in U.S. Pat. No. 7,699,776 B2 to Fuller et al., entitled “Intuitive Ultrasonic Imaging System and Related Method thereof,” filed in the PCT Mar. 6, 2003, which is incorporated herein in its entirety by reference. As discussed in U.S. Pat. No. 7,699,776 B2, in one instance, the components are integrated into a single housing or physical ultrasound device casing that houses the transducer array 102 and the display 118. In this instance, the transducer array 102 and the display 118 are integrated with the system 100 and arranged with respect to each other so that the ultrasound image is displayed over the 2D array such that it is displayed at the location where the image is acquired.

Alternatively, the transducer array 102 is housed in a probe and the remaining components (106-128) are part of a console (e.g., a laptop, a portable device, etc.) or a separate computing system with an integrated and/or separate display. In this configuration, the probe and console have complementary interfaces and communicate with each other, over a hard wired (e.g., a cable) and/or wireless channel, via the interfaces. The console can be supported on a cart or include wheels, being part of a portable US ultrasound imaging apparatus. In another alternative, the console can be affixed or mounted to stationary or static support structure. In these alternative embodiments, more than one probe (e.g., each for a different frequency) can alternately be interfaced with the console for scanning.

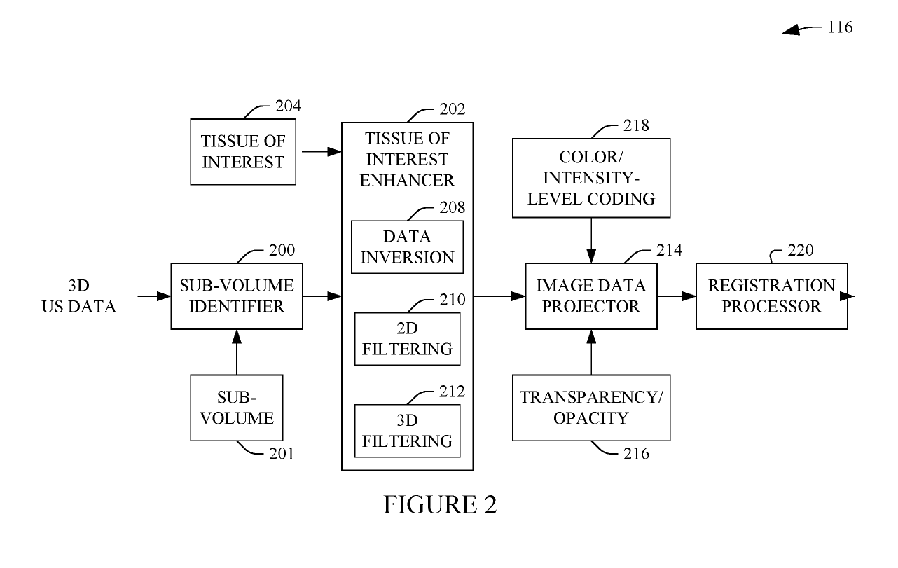

FIG. 2 schematically illustrates a non-limiting example of the 3D image data processor 116.

A sub-volume identifier 200 identifies a sub-volume 201 of the 3D US data for further processing. The sub-volume 201 can be based on a predetermined default sub-volume, a signal indicative of a sub-volume of interest of a user (e.g., received via the user interface 122), a determination of a sub-volume that includes the entire tissue of interest, and/or other approach. By way of non-limiting example, where the 3D US data represents a 5 cm thick volume, the sub-volume identifier 200 can to extract a sub-volume of the 5 cm volume. For instance, the sub-volume identifier 200 can extract a sub-volume 3 cm thick, centered about the center (the 2.5 cm level) of the 5 cm slab. Thus, where tissue of interest is located within a sub-volume of the acquired 3D US data, the sub-volume of the acquired 3D US data including the tissue of interest can be identified and extracted from the 3D US data.

In one instance, the sub-volume is extracted from the 3D US data by applying a weighting function. A suitable weighting function enhances voxels of the sub-volume and/or suppresses voxels outside of the sub-volume. For example, in one instance, the sub-volume identifier 200 applies a Gaussian weighting function to the 3D US data. In another instance, the sub-volume identifier 200 applies a rectangular or other weighting function to the 3D US data. It is to be appreciated that the above example is a non-limiting example. That is, the sub-volume may be other thicknesses, including thinner and thicker sub-volumes. Furthermore, the sub-volume may be centered at another region of the 3D volume, including a lesser or greater depth, relative to the surface of the object adjacent to the transducer array 102.

In another example, the sub-volume identifier 200 is omitted. In this example, the entire 3D US data is further processed as described below.

A tissue of interest enhancer 202 is configured to visually enhance voxels representing a pre-determined tissue of interest 204. By way of example, the illustrated tissue of interest enhancer 202 is configured to enhance voxels via one or more of data inversion 208, 2D filtering 210, 3D filtering 212, a tissue analyzing filter that can analyze the tissue pattern and reconstruct the structure of tissue of interest, and/or other B-mode image data enhancing approaches. One example of these filters is a tensor-based filter which analyzes the tensor of each individual pixel/voxel and the structure around it. Then it performs a tensor eigen value decomposition and the generated eigen values are remapped according to their location and characteristics. The tissue of interest is then reconstructed and enhanced. After 2D/3D filtering, the data can be inverted to high light the flow region (low echogenicity) and suppress other region (high echogenicity).

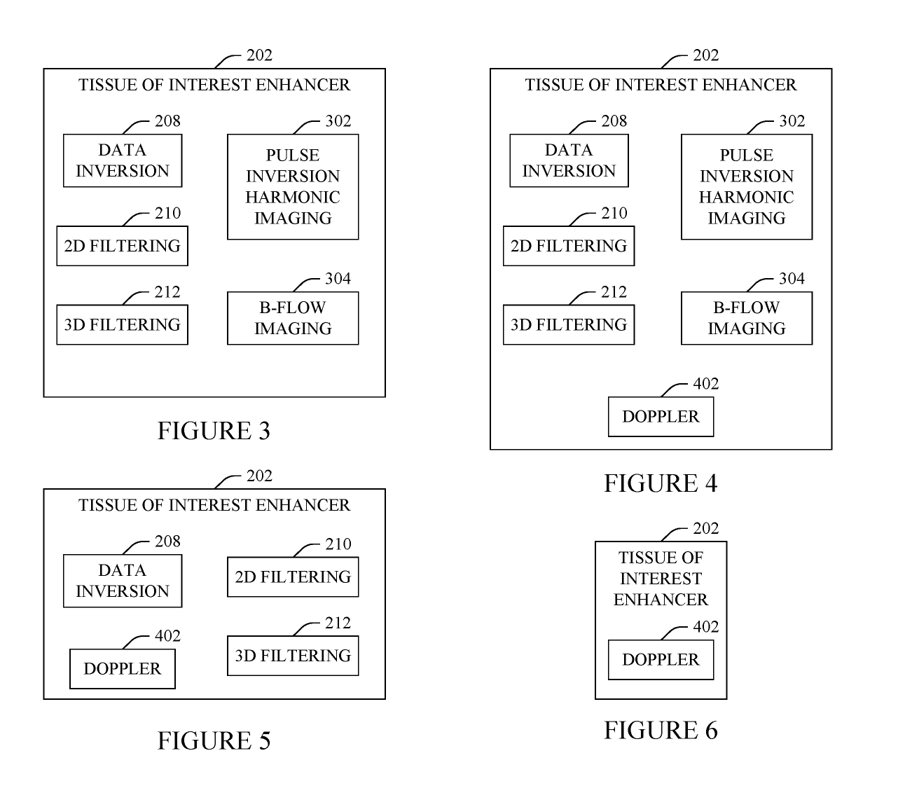

As shown in FIG. 3, in a variation, the tissue of interest enhancer 202 may additionally include non-B-mode imaging enhancing approaches. For example, the variation of FIG. 3 also includes pulse inversion harmonic imaging 302 and B-flow imaging 304, which use stationary echo cancellation techniques. For pulse inversion, two successive pulses of opposite sign are emitted and then subtracted from each other, and with harmonic imaging, a deep penetrating fundamental frequency is emitted and a harmonic overtone is detected. With this approach, noise and artifacts due to reverberation and aberration can be reduced. B-flow imaging directly images blood reflectors providing a real time image of flow that resembles an angiogram. The display can have a simple increase/decrease in gain to optimize a B-Flow image.

As shown in FIG. 4, in another variation, the tissue of interest enhancer 202 also includes Doppler 402 enhancing approaches. In this configuration, the Doppler Effect is used to determine a Doppler signal that can be used to both detect and separate arteries and veins. This can be done, e.g., by identifying a direction and a pulsatility of the flow. FIG. 5 shows a variation with only B-mode (208, 210 and 212) enhancing and the Doppler 402 enhancing. FIG. 6 shows a variation with only the Doppler processing 402. Other variations with similar and/or different, more or less, etc. enhancing approaches are also contemplated herein.

Returning to FIG. 2, an image data projector 214 projects the enhanced 3D US data to 2D or 3D image space through surface or volume rendering approaches. In the illustrated embodiment, the image data projector 214 employs at least one of a transparency/opacity 216, a color/intensity-level coding 218, and/or other algorithm With color/intensity-level coding 218, the image data projector 214 colors and/or intensity codes pixels based on their depth. Such coding differentiates between superficial tissue of interest nearer the surface and deeper tissue of interest. In the presence of the Doppler signal, the colorization could be used to separate pulsatile and none-pulsatile tissue.

With the transparency/opacity algorithm 216, the image data projector 214 sets a transparency of a voxel inversely proportional to its intensity value. In addition, the transparency could be adjusted as a function of imaging depth. For example, in deeper depth, pixel with same intensity value will have more transparency compared with its shallow depth counterparts. This provides an intuitive display of the 3D US data as the signal to noise ratio drops as a function of depth. After assigning the transparency, the image data projector 214 renders the tissue of interest. Surface normals and/or gradient information of the tissue of interest can be extracted and employed during the rendering process to enhance the visualization quality.

A registration processor 220 spatially registers the projected image data with the 2D array the display 118. Generally, this includes spatially registering the projected image data such that the projected image represents the 3D volume right with the 2D array under the surface of the object or subject that is in physical contact with the array 102. This allows the projected image data to be displayed and visualized so that an observer can see the scanned volume, which is the 3D volume right with the 2D array under the surface of the object or subject that is in physical contact with the array, as if the observer is looking directly at the point of contact, without the ultrasound imaging apparatus 100 but with the ability to look through the point of contact and into the volume.

The registration processor 220 may optionally be configured to adjust a point-of-view of the displayed projected image data. For example, in one instance, the registration processor 220 registers the projected image data with the 2D array 102 to visually present a point of view perpendicular to the 2D array 102. This can be done automatically and/or on-demand, e.g., based on a signal transmitted in response to user activation of a control of the interface 122. In another instance, the registration processor 220 registers the projected image data with the 2D array 102 to visually present a point of view a predetermined angle such as 30 degrees with respect to the 2D array 102. In yet another instance, the point of view is dynamically adjustable based on an input signal indicative of an angle of interest of the user. Likewise, dynamic control can be based on a signal transmitted in response to user activation of a control of the interface 122.

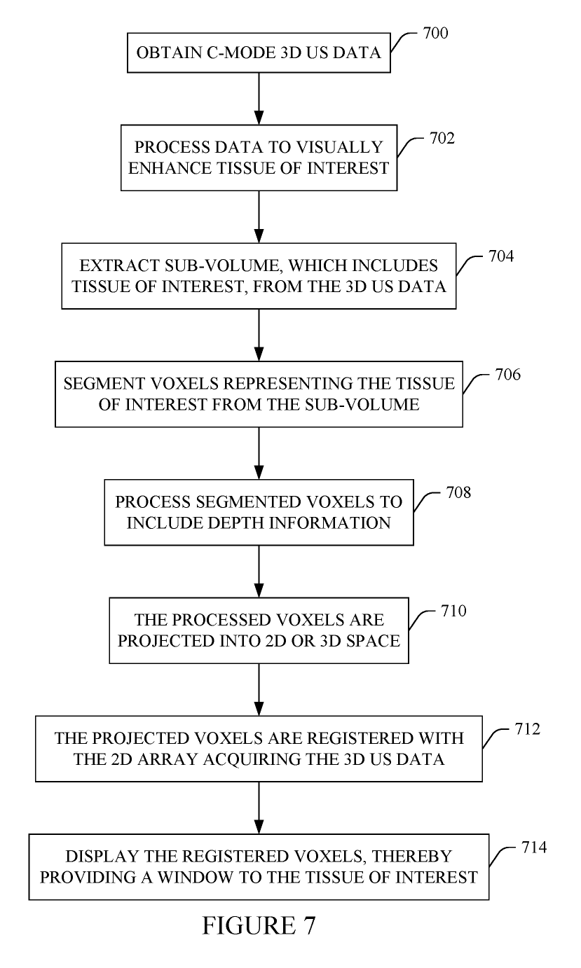

FIG. 7 illustrates an example ultrasound imaging method for processing 3D US data.

It is to be understood that the following acts are provided for explanatory purposes and are not limiting. As such, one or more of the acts may be omitted, one or more acts may be added, one or more acts may occur in a different order (including simultaneously with another act), etc.

At 700, C-mode 3D US data, which includes voxels representing tissue of interest and other tissue, is obtained. The C-mode 3D US data is acquired with a 2D transducer array (e.g., the 2D transducer array 102) of the US imaging apparatus 100 and/or other US imaging apparatus, operating in C-mode.

At 702, the C-mode 3D US data is processed to visually enhance the tissue of interest. In one instance, this includes applying a tissue analyzing filter along with other tissue enhancing methods that can reconstruct and enhance the tissue of interest are performed.

At 704, optionally, a sub-volume of the 3D US data is extracted from the 3D US data. As described herein, a suitable sub-volume includes a plane or planes of voxels that cover the tissue of interest, while excluding a voxels that do not cover the tissue of interest.

At 706, voxels representing the tissue of interest are segmented (e.g., extracted, enhanced, etc.) from the 3D image data. As described herein, this may be through visually enhancing voxels representing the tissue of interest and/or visually suppressing voxels representing the other tissue.

At 708, optionally, the voxels representing the tissue of interest are processed to include depth dependent information. As discussed herein, this may include using opacity/transparency, color/intensity and/or other approaches for adding depth information to image data.

At 710, the voxels representing the tissue of interest are projected into 2D or 3D space via surface or volume rendering.

At 712, the projected voxels are registered with the 2D array 102. As discussed herein, the registration can be such that the point of view is looking into the array 102 at a predetermined angle and can be adjustable, and so that the projected voxels can be displayed as if the display 118 is a window allowing the user to look directly into the 3D US data and see the tissue of interest.

At 714, the registered projected voxels are visually displayed via the display 118 and/or other display. This can be a 2D or a 3D display. As discussed herein, the visual presentation is such that the display effectively becomes a window to the tissue of interest in the subject.

The methods described herein may be implemented via one or more processors executing one or more computer readable instructions encoded or embodied on computer readable storage medium which causes the one or more processors to carry out the various acts and/or other functions and/or acts. Additionally or alternatively, the one or more processors can execute instructions carried by transitory medium such as a signal or carrier wave.

The embodiments described herein can, in one non-limiting instance, be used to visualize vessels such as veins and/or arteries. In this instance, the vascularization under the skin right behind the 2D array is visually enhanced (with respect to the other tissue) and displayed via the display 118. As such, the visualization and the display 118 provides a window through which a user observe see the vascularization under the skin right behind the 2D array.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. An ultrasound imaging apparatus, comprising:
a transducer array configured to acquire a 3D plane of US data parallel to the transducer array, wherein the transducer array includes a 2D array of transducer elements;
a 3D US data processor that visually enhances the structure of tissue of interest and extracts voxels representing tissue of interest therefrom; and
a display, located opposite the transducer array, that displays the extracted voxels representing the tissue of interest the 3D plane of US 3D US data.

2. The apparatus of claim 1, the 3D US data processor, comprising:
a registration processor that spatially registers the extracted voxels with the 2D array of transducer elements.

3. The apparatus of claim 2, wherein the extracted voxels are spatially registered with the 2D array of transducer elements to visually appear to be below an area of contact between the transducer array and an object being scanned.

4. The apparatus of claim 2, wherein the registration processor identifies a view point of the extracted voxels, wherein the view point is perpendicular to the display.

5. The apparatus of claim 2, to wherein the registration processor identifies a view point of the extracted voxels, wherein the view point is not perpendicular to the display.

6. The apparatus of claim 1, the 3D US data processor, comprising:
a tissue of interest enhancer that visually enhances voxels representing the tissue of interest, thereby extracting the voxels representing tissue of interest from the 3D plane of US data.

7. The apparatus of claim 1, the 3D US data processor, comprising:
a tissue of interest enhancer that visually suppresses voxels not representing the tissue of interest, thereby extracting the voxels representing tissue of interest from the 3D plane of US data.

8. The apparatus of claim 6, wherein the 3D US data processor inverts an intensity of the voxels and applies 2D or 3D filtering to the intensity inverted voxels.

9. The apparatus of claim 6, wherein the 3D US data processor generates and utilizes a Doppler signal to identify voxels corresponding to vessels represented in the 3D US data.

10. The apparatus of claim 9, wherein the vessels include veins and arteries, and the 3D US data processor utilizes the Doppler signal to separate veins and arteries based on a direction and a pulsatility of flow.

11. The apparatus of claim 1, the 3D US data processor, comprising:
an image data projector that projects the enhanced voxels into 2D or 3D space.

12. The apparatus of claim 11, wherein the image data projector employs a transparency/opacity to the voxels based voxel intensity value.

13. The apparatus of claim 12, wherein the image data projector further employs a one or more of transparency/opacity, color, or intensity to the voxels based voxel depth within the 3D US data.

14. The apparatus of claim 1, wherein the ultrasound imaging apparatus is a hand-held portable device, and further comprising: a housing that houses the transducer array and the display, wherein the display is mechanically integrated with the housing.

15. The apparatus of claim 1, wherein the 3D US data is C-mode data which includes one or more 3D planes of data, which are parallel to the transducer array.

16. A method, comprising:
obtaining C-mode 3D image data, which includes voxels representing tissue of interest and other tissue;
filtering the C-mode 3D image data to visually enhance the tissue of interest;
segmenting the voxels representing the tissue of interest from the filtered C-mode 3D image data;
projecting the segmented voxels onto a 2D surface or a 3D volume; and
visually displaying the projected segmented voxels so that they tissue of interest appears adjacent to the display.

17. The method of claim 16, further comprising:
spatially registering, prior to displaying the projected segmented voxels, the projected segmented voxels and a transducer array that acquired the C-mode 3D image data.

18. The method of claim 17, wherein the projected segmented voxels represent the tissue of interest directly below the transducer array.

19. The method of claim 16, further comprising:
setting a view point of the displayed projected segmented voxels based on at least one of a default or a user identified view point.

20. The method of claim 19, further comprising:
dynamically adjusting the view point during imaging in response to a signal indicative of a view point of interest of a user.

21. The method of claim 16, the segmenting, comprising:
visually enhancing voxels representing flow.

22. The method of claim 16, the segmenting, comprising:
visually suppressing voxels representing tissue.

23. The method of claim 21, further, comprising:
applying at least one of B-mode or Doppler visual enhancing to visually enhance the voxels representing the tissue of interest.

24. The method of claim 21, further, comprising:
utilizing US data obtained through pulse inversion harmonic imaging to visually enhance the voxels representing the tissue of interest.

25. The method of claim 21, further, comprising:
utilizing US data obtained through B-flow imaging to visually enhance the voxels representing the tissue of interest.

26. The method of claim 21, further, comprising:
utilizing US data obtained through Doppler imaging to separate veins and arteries based on a direction and a pulsatility of flow.

27. The method of claim 16, the projecting, comprising:
assigning a transparency/opacity to each voxel based on a corresponding voxel intensity value.

28. The method of claim 27, the projecting, comprising:
assigning at least one of a transparency/opacity or a colo/intensity to each voxel based on a depth of each voxel in the C-mode 3D imaging data.

29. The method of claim 16, further, comprising:
extracting a sub-volume of the C-mode 3D image data; and
segmenting the voxels representing the tissue of interest from the sub-volume.

30. The method of claim 29, further, comprising:
applying a weighting function to the 3D plane of US data to extract the sub-volume.

31. A computer readable storage medium encoded with computer readable instructions, which, when executed by a processer, causes the processor to:
acquire 3D US imaging data with voxels representing tissue of interest and other tissue, wherein the 3D US imaging data is C-mode data;
visually enhance the structure of tissue of interest through filtering;
extract the voxels representing the tissue of interest from the filtered 3D US imaging data;
at least one of surface or volume render the extracted voxels; and
register the rendered voxels with a 2D array the acquired the 3D US imaging data; and display the registered voxels.

32. The computer readable storage medium of claim 31, wherein the computer readable instructions, which, when executed by the processer, further causes the processor to:
prior to extracting the tissue of interest, identify a sub-volume of the 3D US data to extract the tissue of interest from; and
prior to projecting the voxels, process the voxels to add depth information to the voxels.
